(19) United States
(12) Patent Application Publication
(10) Pub. No.: US 2020/0376452 A1
(43) Pub. Date: Dec. 3, 2020

(54) Device for Optimization of Microorganism Growth in Liquid Culture

(71) Applicants: MicrobeDX, Inc., Pacific Palisades, CA (US); The Regents of the University of California, Oakland, CA (US)

(72) Inventors: Alexandra Perebikovsky, Irvine, CA (US); Bernard Churchill, Los Angeles, CA (US); Scott Churchman, Santa Monica, CA (US); David Arnold Haake, Culver City, CA (US); Colin Wynn Halford, Los Angeles, CA (US); Yujia Liu, Irvine, CA (US); Marc Madou, Irvine, CA (US); Gabriel Monti, Cypress, CA (US)

(21) Appl. No.: 16/641,793

(22) PCT Filed: Aug. 30, 2018
(86) PCT No.: PCT/US2018/048906

§ 371 Date: Feb. 25, 2020

Related U.S. Application Data
(60) Provisional application No. 62/552,332, filed on Aug. 30, 2017.

(51) Int. Cl. B01F 11/00; C12M 3/06; B01L 3/00

(52) U.S. Cl. CPC B01F 11/0062; C12M 27/14; B01L 2300/0803; B01F 2215/0454; B01L 3/5085

(57) ABSTRACT

There is described a system for growing a microorganism in liquid culture, the system comprising: a driving apparatus configured to house and oscillate a microfluidic cartridge; and a microfluidic cartridge comprising at least one incubation chamber, such that when the system is in use, the incubation chamber may be oscillated back and forth along an oscillation path using a preferred oscillation protocol. There is also described a method of growing a microorganism in liquid culture, the method comprising disposing a microorganism and suitable growth medium into an incubation chamber; and mixing the microorganism and growth medium by oscillating the incubation chamber back and forth along an oscillation path using a preferred oscillation protocol. There is also described a microfluidic cartridge that may be used to grow microorganisms using the system and methods described above.

### CROSS-REFERENCE TO RELATED APPLICATION

The present application claims the benefit under 35 U.S.C. § 119(e) of provisional patent application Ser. No. 62/552,332, filed Aug. 30, 2017, the contents of which are hereby incorporated by reference.

### BACKGROUND OF THE INVENTION

### Field of the Invention

In one of its aspects, the present invention relates to a system for growing a microorganism in liquid culture. In another of its aspects, the present invention relates to a method of growing a microorganism in liquid culture. In yet another of its aspects, the present invention relates to a microfluidic cartridge that may be used to grow microorganisms using the system and methods disclosed herein.

### Description of the Prior Art

For conventional assays involving pathogenic bacteria, the step of incubating to induce bacterial growth is often rate limiting, typically taking hours to days and requiring transport to a central lab. The long lead times may have deleterious effects. For example, in antibiotic susceptibility testing, slow testing often leads to “best guess” methods to determine treatment options, which contributes to antibiotic resistance. Furthermore, traditional liquid bacteria cultures are grown in 96-well plates which requires the use of bulky and expensive plate shakers.

Accordingly, it would be desirable to have an improved system and methods for rapidly growing microorganisms in liquid culture. It would also be desirable for this improved system to be portable and more cost effective than the microorganism growth systems previously used in the art.

### SUMMARY OF THE INVENTION

It is an object of the present invention to obviate or mitigate at least one of the above-mentioned disadvantages of the prior art.

It is another object of the present invention to provide a novel system, methods and apparatus for improving the rate of growth of microorganisms in liquid culture.

Accordingly, in one of its aspects, the present invention provides a system for growing a microorganism in liquid culture, comprising:

(a) a rotating platform on a driving apparatus; and

(b) at least one cartridge comprising a plurality of incubation chambers which rests upon the rotating platform, wherein said rotating platform provides vortical flow and/or turbulent mixing within the plurality of incubation chambers.

In another of its aspects, the present invention provides a system for growing a microorganism in liquid culture, comprising:

(a) a driving apparatus configured to house and oscillate a microfluidic cartridge; and

(b) a microfluidic cartridge secured with respect to the driving apparatus, the microfluidic cartridge comprising: a body portion and at least a first incubation chamber comprising (i) a first wall, (ii) a second wall opposed to the first wall, and (iii) at least one sidewall interconnecting the first wall and the second wall to define a chamber interior having a chamber volume and configured to contain a liquid, wherein a ratio of the first wall surface area to chamber volume is at least about 19 mm⁻¹;

wherein at least a portion of at least one of the first wall and second wall is gas permeable.

In yet another of its aspects, the present invention provides a method for growing a microorganism in a liquid culture comprising:

(a) disposing a microorganism and a suitable growth medium in a first incubation chamber, wherein the incubation chamber comprises (i) a first wall, (ii) a second wall opposed to the first wall, and (iii) at least one sidewall interconnecting the first wall and the second wall to define a chamber interior having a chamber volume and configured to contain a liquid, wherein a ratio of the first wall surface area to chamber volume is at least about 19 mm⁻¹, wherein at least a portion of at least one of the first wall and second wall is gas permeable; and

(b) mixing the microorganism and the growth medium by oscillating the incubation chamber back and forth along an oscillation path at a predetermined oscillation frequency.

In yet another of its aspects, the present invention provides a microfluidic cartridge comprising:

(a) a body portion having a mounting portion configured to be secured with respect to a driving apparatus;

(b) at least a first incubation chamber disposed in the body portion of the first incubation chamber comprising (i) a first wall, (ii) a second wall opposed to the first wall, and (iii) at least one sidewall interconnecting the first wall and the second wall to define a chamber interior having a chamber volume and configured to contain a liquid, wherein a ratio of the first wall surface area to chamber volume is at least about 19 mm⁻¹;

wherein at least a portion of at least one of the first wall and second wall is gas permeable.

In yet another of its aspects, the present invention provides a microfluidic cartridge used for growing a microorganism in liquid culture comprising:

(a) a body portion having a mounting portion configured to be secured with respect to a driving apparatus;

(b) at least a first incubation chamber disposed in the body portion of the first incubation chamber and configured so that when the microfluidic cartridge is in use and engaged by the driving apparatus, the first incubation chamber is translated back and forth along an oscillation path at a predetermined oscillation frequency, creating turbulent mixing within the first incubation chamber, wherein, the first incubation chamber comprises (i) a first wall, (ii) a second wall opposed to the first wall, and (iii) at least one sidewall interconnecting the first wall and the second wall to define a chamber interior having a chamber volume and configured to contain a liquid, wherein a ratio of the first wall surface area to chamber volume is at least about 19 mm⁻¹;

wherein at least a portion of at least one of the first wall and second wall is gas permeable to facilitate a flow of gas into and out of the incubation chamber.

Accordingly, as described herein below, the present inventors have developed a system and methods for rapid, on-site growth of microorganisms in liquid culture that is faster, less bulky and more cost efficient than traditional growth techniques.

For liquid bacterial cultures, rapid and healthy growth depends on factors including (1) sample aeration, so that bacteria samples have access to atmospheric gases (e.g., oxygen) for growth, (2) nutrient availability, where samples are thoroughly mixed to provide nutrients homogenously throughout the culture, and (3) minimization of biofilms and clumping, where shaking and agitation prevents bacteria culture from settling to the bottom of a chamber and forming biofilms or clumps that hinder reproduction.

To address the challenges of the conventional art, these principles are applied in designing a portable microorganism growth system which includes a rotatable microfluidic cartridge that is used in conjunction with an oscillation driving apparatus and an oscillation protocol optimized for mixing liquid bacterial samples.

In order to provide access to atmospheric gases (e.g., oxygen) to increase growth, the present inventors have developed a portable rotatable microfluidic cartridge that is specifically designed to increase sample aeration in several ways. First, the microfluidic cartridge contains an incubation chamber with at least one gas permeable membrane that facilitates the flow of gas into and out of the incubation chamber during the incubation process. This gas flow generates bubbles within the incubation chamber, providing more surface area for gas exchange within the sample during mixing. Second, the surface area to volume ratio of the incubation chamber, (where the surface area of the chamber is measured in the same plane as the direction of rotation of the rotating microfluidic cartridge) is configured to be larger than that of traditional 96-well plates, in order to allow for more turbulence in the incubation chamber during mixing and further to allow for better gas exchange through the gas permeable membrane. Traditional 96-well plates may for example have a surface area to volume ratio of about 19 mm⁻¹. The microfluidic cartridges developed by the present inventors thus have a surface area to volume ratio that is larger than that of traditional 96-well plates. For example, incubation chambers of the microfluidic cartridges disclosed herein may have a surface area to volume ratio of at least 19 mm⁻¹. Finally, the growth system is designed so that the incubation chamber is intended to be only partially filled with a liquid sample, leaving a head space of air in the sample during mixing. This headspace provides further aeration to the sample. While not wishing to be bound by any particular theory or mode of action, it is believed that the above-mentioned features of the microfluidic cartridge design facilitate optimal amounts of aeration to allow for increased microorganism growth.

In order to ensure thorough mixing, to provide nutrients homogenously throughout the culture, and to minimize the formation of biofilms and clumping during bacterial growth, the present inventors have developed a system with an optimized mixing protocol to be used on a microfluidic cartridge. Traditional microfluidic systems have low Reynolds numbers and exhibit laminar flow regimes, which are dominated by viscous, rather than inertial forces. Thus, without turbulent mixing, microfluidic devices must rely on either passive molecular diffusion or external energy sources. Furthermore, the small, enclosed volumes characteristic of microfluidic systems restrict access of the bacterial culture to fresh oxygen and other atmospheric gases, making sample aeration difficult without bulky or complex pumps that bubble gases from an external source. By combining a rotatable microfluidic cartridge with an oscillation driving apparatus, the present inventors have developed an efficient method for mixing a bacterial sample within a microfluidic system. In this system, the oscillating driving apparatus creates a Euler force that results in chaotic advection and turbulent mixing of bacteria samples at a higher rate than in a 96-well plate or culture flask method. In an oscillating system, the Euler force (which is perpendicular to centrifugal force), may be used to generate vortical flow and/or provide uniform turbulent mixing within a microfluidic chamber of the microfluidic system. Euler forces are inertial forces that are produced when the microfluidic system (i.e., an incubation chamber) experiences cycles of unidirectional acceleration-and-deceleration rotation. Thus, mixing is influenced by chamber geometry, orientation, acceleration/deceleration rate, and angular spin. For example, as disclosed herein, the incubation chambers comprise three dimensions: length, width and depth. In certain embodiments, the length may be oriented tangentially to the direction of rotation of the cartridge. The microfluidic cartridges developed by the present inventors have been designed such that each of these dimensions allows for increased turbulent mixing within the chamber when the cartridge is rotated or oscillated. For example, the incubation chamber may be configured so that the length is greater than the width, and the length and width are each significantly greater than the depth. Further, as highlighted above, the surface area (measured in the same plane as the direction of rotation of the rotating microfluidic cartridge and calculated based on the length and width of the chamber) may be configured such that ratio of the surface area to chamber volume is larger than that of traditional 96-well plates. While not wishing to be bound by any particular theory or mode of action, it is believed that by manipulating the dimensions of the incubation chamber in this way, the microfluidic cartridge design facilitates turbulent mixing within the chamber to allow for increased microorganism growth.

Unlike traditional liquid bacteria cultures grown in 96-well plates in bulky and expensive plate shakers, the oscillation driving apparatus and microfluidic cartridge described above represent an inexpensive and portable alternative that yields faster growth of bacteria. This system may be used to either increase signal of an existing assay or to decrease assay time by achieving a measurable signal faster. In one exemplary application, the rotatable microfluidic cartridge and oscillation driving apparatus may be used in developing ultrafast, point of care antibiotic susceptibility assays which require rapid culture of bacteria with different antibiotics to determine resistance.

As illustrated through experimental data hereinbelow, the present inventors have shown that the use of a rotating microfluidic cartridge in conjunction with an oscillation driving apparatus yields superior bacterial growth rates compared with traditional shaker incubators, including 96-well plates on a shaker.

### BRIEF DESCRIPTION OF THE DRAWINGS

Embodiments of the present invention will be described with reference to the accompanying drawings, wherein like reference numerals denote like parts, and in which:

FIG. 1 illustrates an interior view of an oscillation driving apparatus housing a microfluidic incubation cartridge on a spin-chuck with a DC motor integrated into a metal heater.

FIG. 2 illustrates a fully assembled microfluidic incubation cartridge, in accordance with some aspects of the present disclosure.

FIG. 3 illustrates an exemplary microfluidic cartridge, in accordance with some aspects of the present disclosure.

FIG. 4 illustrates a microfluidic cartridge, including an exemplary oscillation path and oscillation protocol, in accordance with some aspects of the present disclosure.

FIG. 5 illustrates a cross-sectional view of an exemplary incubation chamber in a microfluidic cartridge, in accordance with some aspects of the present disclosure.

FIG. 6 illustrates an exploded view of a microfluidic incubation cartridge assembly.

FIGS. 7A and 7B illustrate a comparison of E. coli growth dynamics in a 96-well plate in shaker, an incubation cartridge in shaker, and an incubation cartridge in spin-stand incubator through 90 minutes of 37° C. incubation. FIG. 7A provides results in the form of bacterial growth in CFU/mL and FIG. 7B provides results in the form of Luminex signal strength (which can be directly correlated to bacterial growth in CFU/mL). In both FIGS. 7A and 7B the solid line represents E. coli growth in an incubation cartridge in a spin-stand incubator; the dotted line represents E. coli growth in an incubation cartridge on a shaker; and the dashed line represents E. coli growth in a 96-well plate on a shaker.

FIGS. 8A and 8B illustrate a comparison of bacterial growth using a gas-permeable membrane and a non-gas permeable membrane in an incubation cartridge in spin-stand incubator, in accordance with some aspects of the present disclosure. FIG. 8A shows the resulting Luminex signal results for bacteria grown in a microfluidic cartridge without a permeable membrane compared to a 96-well plate in shaker, while FIG. 8B shows the resulting Luminex signal results for bacteria grown in a microfluidic cartridge with a gas permeable membrane compared to a 96-well plate in shaker.

FIG. 9 provides tabular results showing an improvement in bacterial growth rates in an incubation cartridge in spin-stand incubator for several different antibiotic resistant microorganisms in antibiotic infused samples compared to a 96-well plate in shaker.

### DETAILED DESCRIPTION OF THE PREFERRED EMBODIMENTS

The present invention relates to a system for growing a microorganism in liquid culture, comprising a rotating platform on a driving apparatus; and at least one cartridge comprising a plurality of incubation chambers which rests upon said rotating platform, wherein said rotating platform provides vortical flow and/or turbulent mixing within the plurality of incubation chambers.

In another of its aspects, the present invention relates to a system for growing a microorganism in liquid culture, comprising: (a) a driving apparatus configured to house and oscillate a microfluidic cartridge; and (b) a microfluidic cartridge secured with respect to the driving apparatus, the microfluidic cartridge comprising: a body portion and at least a first incubation chamber comprising (i) a first wall, (ii) a second wall opposed to the first wall, and (iii) at least one sidewall interconnecting the first wall and the second wall to define a chamber interior having a chamber volume and configured to contain a liquid, a ratio of the first wall surface area to chamber volume ratio is at least about 19 mm⁻¹; wherein at least a portion of at least one of the first wall and second wall is gas permeable to facilitate a flow of gas into and out of the chamber interior.

Preferred embodiments of this system may include any one or a combination of any two or more of any of the following features:
the microfluidic cartridge is a circular disc;
the incubation chamber has a curved, rectilinear, curvilinear or wedge shape;
the first wall of the incubation chamber is gas permeable;
the second wall of the incubation chamber is gas permeable;
the first wall of the incubation chamber comprises a breathable membrane;
the second wall of the incubation chamber comprises a breathable membrane;
the breathable membrane is fabricated from a copolymer, such as polyester-polyurethane or polyether-polyurethane;
the breathable membrane is comprised of a biocompatible polymer film that is gas permeable and liquid and microbe impermeable;
the first wall of the incubation chamber is configured to permit a flow of gas into and out of the incubation chamber;
the second wall of the incubation chamber is configured to permit a flow of gas into and out of the incubation chamber;
the microfluidic cartridge comprises a plurality of incubation chambers;
the plurality of incubation chambers is integrally disposed in a common body portion of the cartridge;
the plurality of incubation chambers is disposed annularly around a central axis on the cartridge;
the plurality of incubation chambers is configured to oscillate in unison about a central axis on the cartridge;
the plurality of incubation chambers are fluidically isolated from one another;
the microfluidic cartridge further comprises at least one additional processing chamber disposed in the body of the cartridge;
the additional processing chamber is connected to the incubation chamber by a microfluidic path and is located either upstream or downstream from the incubation chamber;
the body of the cartridge comprises a polymer, wherein the polymer is selected from poly(methyl methacrylate) (PMMA), polycarbonate, polyethylene, polypropylene, polystyrene, polyesters, polyvinyl chloride (PVC), cyclic olefin polymer (COP), cyclic olefin copolymer (COC) and nylon;
the driving apparatus is configured to oscillate the microfluidic cartridge in an arcuate oscillation path;
the driving apparatus is configured to oscillate the microfluidic cartridge at an oscillation angle of about 180 degrees;
the driving apparatus is configured to oscillate the microfluidic cartridge at an oscillation frequency of between 1n and 5 Hz, or more specifically 2 Hz or 4 Hz;
the driving apparatus is configured to oscillate the microfluidic cartridge in a linear oscillation path;
the driving apparatus is configured to oscillate the microfluidic cartridge at an angular acceleration in a range between 100 to 500 rad/s²;
the system further includes an incubator comprising a heating element;
the heating element is made of metal, such as Ni/Cr, Cu/Ni or Fe/Cr/Al.

In yet another of its aspects, the present invention relates to a method for growing a microorganism in a liquid culture comprising: (a) disposing a microorganism and a suitable growth medium in a first incubation chamber, wherein the incubation chamber comprises (i) a first wall, (ii) a second wall opposed to the first wall, and (iii) at least one sidewall interconnecting the first wall and the second wall to define a chamber interior having a chamber volume and configured to contain a liquid, wherein a ratio of the first wall surface area to chamber volume is at least about 19 mm⁻¹, wherein at least a portion of at least one of the first wall and second wall is gas permeable; and (b) mixing the microorganism and the growth medium by oscillating the incubation chamber back and forth along an oscillation path at a predetermined oscillation frequency.

Preferred embodiments of this method may include any one or a combination of any two or more of any of the following features:
the method is further includes incubating the microorganism by placing the incubation chamber in an incubator;
the incubator comprises a heating element;
the heating element is made of metal, such as Ni/Cr, Cu/Ni or Fe/Cr/Al;
the method further comprises disposing a microorganism and a suitable growth medium in at least one additional incubation chamber;
the growth medium of one of the incubation chambers comprises an anti-microbial free cell culture medium, while the growth medium of at least one other incubation chamber comprises an anti-microbial agent;
the anti-microbial agent is an antibiotic;
the method further comprises incubating the micro-organism in a bacterial growth broth solution;
the bacterial growth broth solution is a cation-adjusted broth, such as Mueller Hinton broth, lysogeny broth, super optimal broth, super optimal broth with catabolite repression, terrific broth, or M9 minimal broth;
the method further comprises introducing oxygen into the chamber by passing oxygen through a gas permeable portion of either the first wall or the second wall of the chamber;
the oscillation path is arcuate
the oscillation angle is between 100 and 260 degrees, or more preferably is around 180 degrees;
the oscillation frequency is between 1n and 5 Hz, or more specifically 2 Hz or 4 Hz;
the oscillation path is linear;
the angular acceleration is between 100 to 500 rad/s²;
the microorganism is a bacterium;
the bacterium is gram-negative or gram-positive;
the microorganism is fungal; and
the microorganism and suitable growth medium occupy no more than ⅔ of the volume of the incubation chamber, creating a headspace in the chamber.

In yet another of its aspects, the present invention relates to a microfluidic cartridge used for growing a microorganism in liquid culture comprising: (a) a body portion having a mounting portion configured to be secured with respect to a driving apparatus; and (b) at least a first incubation chamber disposed in the body portion of the first incubation chamber comprising (i) a first wall, (ii) a second wall opposed to the first wall, and (iii) at least one sidewall interconnecting the first wall and the second wall to define a chamber interior having a chamber volume and configured to contain a liquid, a ratio of the first wall surface area to chamber volume ratio is at least about 19 mm⁻¹; wherein at least a portion of at least one of the first wall and second wall is gas permeable.

Preferred embodiments of this apparatus may include any one or a combination of any two or more of any of the following features:
the microfluidic cartridge is a circular disc;
the incubation chamber has a curved, rectilinear, curvilinear or wedge shape;
the first wall of the incubation chamber is gas permeable;
the second wall of the incubation chamber is gas permeable;
the first wall of the incubation chamber comprises a breathable membrane;
the second wall of the incubation chamber comprises a breathable membrane;
the breathable membrane is fabricated from a copolymer, such as polyester-polyurethane or polyether-polyurethane;
the breathable membrane is comprised of a biocompatible polymer film that is gas permeable and liquid an microbe impermeable;
the first wall of the incubation chamber is configured to permit a flow of gas into and out of the incubation chamber;
the second wall of the incubation chamber is configured to permit a flow of gas into and out of the incubation chamber;
the microfluidic cartridge comprises a plurality of incubation chambers;
the plurality of incubation chambers is integrally disposed in a common body portion of the cartridge;
the plurality of incubation chambers is disposed annularly around a central axis on the cartridge;
the plurality of incubation chambers is configured to oscillate in unison about a central axis on the cartridge;
the plurality of incubation chambers are fluidically isolated from one another;
the microfluidic cartridge further comprises at least one additional processing chamber disposed in the body of the cartridge;
the additional processing chamber is connected to the incubation chamber by a microfluidic path and is located either upstream or downstream from the incubation chamber;
the body of the cartridge comprises a polymer, wherein the polymer is selected from poly(methyl methacrylate) (PMMA), polycarbonate, polyethylene, polypropylene, polystyrene, polyesters, polyvinyl chloride (PVC), cyclic olefin polymer (COP), cyclic olefin copolymer (COC) and nylon.

As used herein, certain terms may have the following defined meanings.

As used in the specification and claims, the singular form “a,” “an” and “the” include singular and plural references unless the context clearly dictates otherwise. For example, the term “a cell” includes a single cell as well as a plurality of cells, including mixtures thereof

As used in the specification and claims, the term “RiboGrow™” refers to the use of a rotating platform system, as described herein, for increasing growth of a cell, such as a microorganism, in a liquid culture. For instance, a RiboGrow™ method for increasing growth of a microorganism in a liquid culture may be based on placing a cell culture medium comprising at least one microorganism in at least one chamber of a cartridge comprising a plurality of incubation chambers, the liquid within the plurality of incubation chambers of said cartridge being sealed within the chambers by a breathable membrane; rotating the cartridge to generate vortical flow and/or turbulent mixing within the plurality of incubation chambers; and incubating the rotating cartridge at a temperature optimized to induce growth of the microorganism.

As used herein, the term “cell culture media,” refers to a media where a microorganism is capable of rapid growth.

As used herein, the term “breathable membrane” refers to a membrane that is pervious to gases and impervious to liquids as well as microorganisms. In some embodiments, a breathable membrane is a bio-compatible polymer film.

### Systems for Increasing Microorganism Growth Rates in Culture

Disclosed herein are systems for growing a microorganism in liquid culture. Systems for growing a microorganism in liquid culture may comprise (a) a rotating platform on a driving apparatus; and (b) at least one cartridge comprising a plurality of incubation chambers which rests upon said rotating platform, wherein said rotating platform provides turbulent mixing within the plurality of incubation chambers.

As further disclosed herein, systems for growing a microorganism in liquid culture may comprise (a) a driving apparatus configured to house and oscillate a microfluidic cartridge; and (b) a microfluidic cartridge secured with respect to the driving apparatus, the microfluidic cartridge comprising: a body portion and at least a first incubation chamber comprising (i) a first wall, (ii) a second wall opposed to the first wall, and (iii) at least one sidewall interconnecting the first wall and the second wall to define a chamber interior having a chamber volume and configured to contain a liquid, wherein a ratio of the first wall surface area to chamber volume is at least about 19 mm⁻¹; wherein at least a portion of at least one of the first wall and second wall is gas permeable to facilitate a flow of gas into and out of the chamber interior.

### Microfluidic Incubation Cartridge

In one of its aspects, the present invention provides a microfluidic cartridge for growing a microorganism in liquid culture. The microfluidic cartridge may comprise (a) a body portion having a mounting portion configured to be secured with respect to a driving apparatus; and (b) at least a first incubation chamber disposed in the body portion of the first incubation chamber comprising (i) a first wall, (ii) a second wall opposed to the first wall, and (iii) at least one sidewall interconnecting the first wall and the second wall to define a chamber interior having a chamber volume and configured to contain a liquid, wherein a ratio of the first wall surface area to chamber volume is at least about 19 mm⁻¹; wherein at least a portion of at least one of the first wall and second wall is gas permeable.

In certain preferred embodiments, the mounting portion of body of the microfluidic cartridge may be configured to allow the cartridge to remain secured to the driving apparatus when the cartridge oscillates at a predetermined angular acceleration with a predetermined oscillation angle.

In certain preferred embodiments, the incubation chamber of the microfluidic cartridge may be configured so that when the microfluidic cartridge is in use and engaged by a driving apparatus, the first incubation chamber is translated back and forth along an oscillation path at a predetermined oscillation frequency, creating turbulent mixing within the first incubation chamber. In certain embodiments, turbulent mixing within the first incubation chamber may be accomplished as a result of the design of the incubation chamber. For example, the incubation chamber may be designed such that the ratio of the first wall surface area to chamber volume is at least greater than that of traditional 96-well plates. By way of non-limiting example, traditional 96-well plates may have a surface area to volume ratio of about 19 mm⁻¹. By using an incubation chamber designed to include a surface area to volume ratio of at least 19 mm⁻¹, the present invention may facilitate greater mixing capabilities than achievable by traditional 96-well plates and thus may facilitate higher growth rates of microorganisms in said incubation chambers than on 96-well plates. In certain preferred embodiments, the ratio of the first wall surface area to chamber volume may be at least greater than about 20 mm⁻¹ or greater than about 25 mm⁻¹ or greater than about 30 mm⁻¹ or greater than about 35 mm⁻¹ or greater than about 40 or greater than about 45 mm⁻¹ or greater than about 50 mm⁻¹.

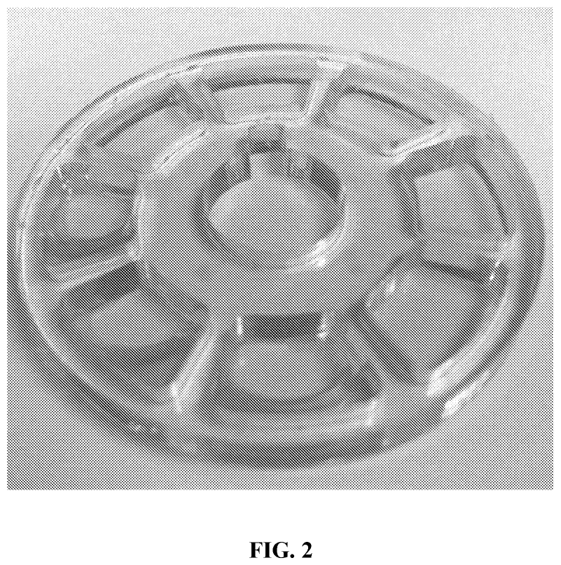

FIGS. 2-4 and 6 show exemplary microfluidic cartridges, according to some aspects of the present disclosure and are discussed in detail below. While these figures illustrate some examples of combinations and configurations of certain features of the present invention, it is understood that other combinations and configurations of these features are also encompassed herein.

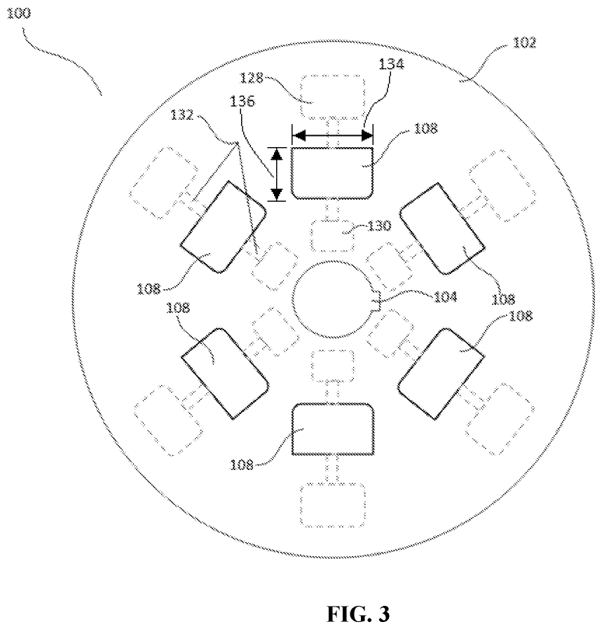

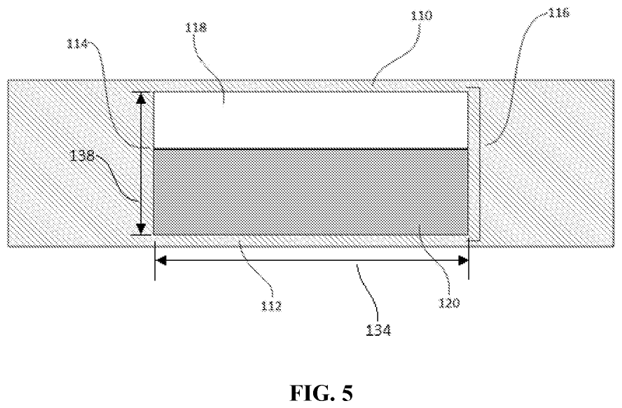

FIG. 3 provides an exemplary microfluidic cartridge 100, in accordance with some aspects of the present disclosure. While in certain preferred embodiments, as illustrated in FIG. 3, the microfluidic cartridge 100 may comprise a circular disc, in other embodiments, the microfluidic cartridge may comprise a non-circular shape. As shown in FIG. 3, in certain preferred embodiments, the microfluidic cartridge 100 may comprise a body portion 102 having a mounting portion 104 which is configured to be secured with respect to a driving apparatus. The microfluidic cartridge may also comprise at least a first incubation chamber 108 disposed in the body portion 102. Each incubation chamber 108 may comprise three dimensions: a length 134, a width 136 and a depth 138 (See FIG. 5). As outlined above, in certain embodiments, it may be desirable for the incubation chamber to have a ratio of the first wall surface area to chamber volume of at least about 19 mm⁻¹. By way of non-limiting example, in one embodiment, the present inventors have developed a microfluidic cartridge to grow bacteria using the system and methods described herein, where the incubation chamber has a first wall surface area (calculated as a factor of chamber length 134 and width 136) of 94 mm² and a chamber volume of 184 mm³, with a resulting surface area to volume ratio of 51 mm⁻¹.

As further shown in FIG. 3, the microfluidic cartridge may comprise a plurality of incubation chambers 108. In certain preferred embodiments, each of the plurality of incubations chambers may be disposed annularly around a central axis on the microfluidic cartridge 100, and more preferably, each of the plurality of incubation chambers may be configured to oscillate in unison about a central axis when the microfluidic cartridge is oscillated. As illustrated in FIG. 3, in certain preferred embodiments, the plurality of incubation chambers may be fluidically isolated from one another. In some embodiments, the cartridge may comprise 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 or more incubation chambers.

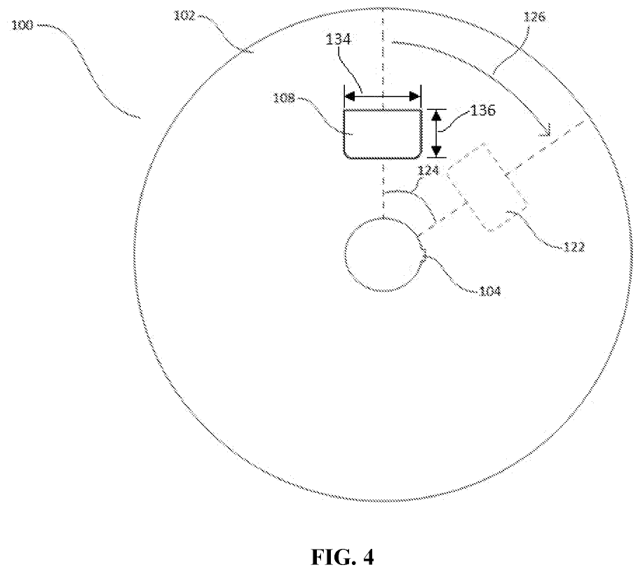

FIG. 4 illustrates one embodiment of a microfluidic cartridge 100, including an exemplary oscillation path 126, in accordance with some aspects of the present disclosure, and FIG. 5 illustrates a cross-sectional view providing a more detailed view of an exemplary incubation chamber in a microfluidic cartridge, in accordance with some aspects of the present disclosure. As shown in FIGS. 4 and 5, each incubation chamber 108 has three dimensions: a length 134, a width 136 and a depth 138. In certain embodiments, the incubation chamber 108 may be oriented on the microfluidic cartridge 100 such that the length of the chamber 134 is aligned tangentially with the oscillation path 126. Such orientation is illustrated in FIG. 4. In other embodiments, the incubation chamber 108 may be oriented on the microfluidic cartridge 100 such that the width of the chamber 136 is aligned tangentially with the oscillation path 126. As illustrated in FIGS. 4 and 5, the incubation chamber may be shaped such that chamber length 134 is larger than the chamber width 136, and that both the chamber length 134 and chamber width 136 are substantially larger than the chamber depth 138. This incubation chamber shape may facilitate turbulent mixing when the chamber is oscillated in the direction of the oscillation path 126. Other embodiments may comprise different chamber geometries and orientations than those illustrated in FIGS. 4 and 5.

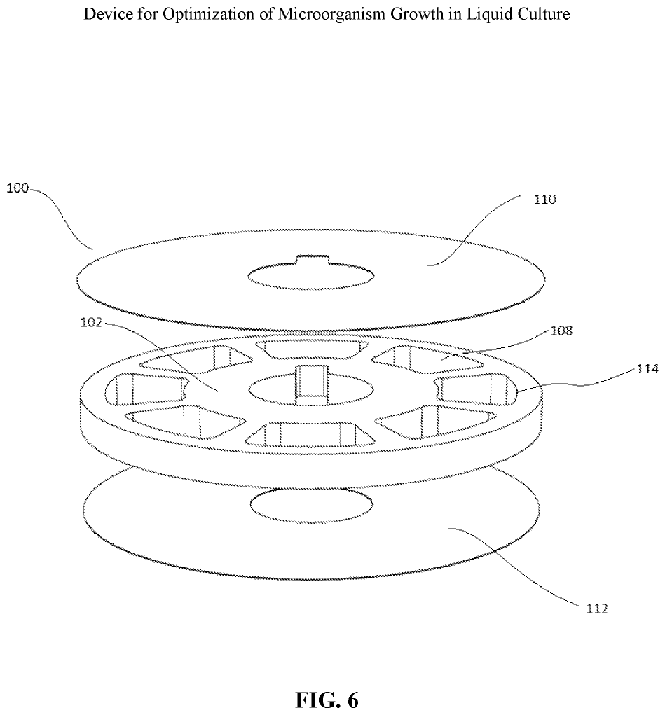

FIG. 6 illustrates an exploded view of one embodiment of a microfluidic cartridge assembly with a plurality of incubation chambers 108 disposed on the body portion 102 of the cartridge 100, wherein each incubation chamber 108 comprises a first wall 110, a second wall 112, and at least one sidewall 114 interconnecting the first wall 110 and the second wall 112 to define a chamber interior.

In some embodiments, the cartridge may have a diameter in the range of about 30 mm or about 40 mm or about 50 mm or about 60 mm or about 70 mm or about 80 mm or about 90 mm or about 100 mm or about 110 mm to about 120 mm or about 130 mm or about 140 mm or about 150 mm or about 160 mm or about 170 mm or about 180 mm or about 190 mm or about 200 mm. In some embodiments, the cartridge may have a diameter sufficient to be portable and/or easy to handle. For example, the cartridge may be as small as 30 mm and still be easy to hold and as large as 200 mm and still be portable. When the cartridge diameter is smaller than 30 mm, the cartridge may be difficult to handle. When the cartridge diameter is larger than 200 mm, the cartridge may be difficult to transport. In certain preferred embodiments, the cartridge may have a diameter of approximately 120 mm.

As further shown in FIG. 3, the microfluidic cartridge 100 may further comprise at least one additional processing chamber 128 or 130 disposed in the body portion 102 of the microfluidic cartridge 100. In certain preferred embodiments, the additional processing chamber may be connected to the first incubation chamber by a microfluidic pathway 132 on the microfluidic cartridge 100. By way of non-limiting example, in certain embodiments, the additional processing chamber 130 may be located upstream from the first incubation chamber 108. In other embodiments, the additional processing chamber 128 may be located downstream from the first incubation chamber 108. In some embodiments, the cartridge is configured so that further processing occurs within the incubation chamber itself.

FIG. 5 illustrates a cross-sectional view providing a more detailed view of an exemplary incubation chamber in a microfluidic cartridge, in accordance with some aspects of the present disclosure. A shown in FIG. 5, in certain preferred embodiments, the first incubation chamber 108 may comprise a first wall 110, a second wall 112 opposed to the first wall, and at least one sidewall 114 interconnecting the first wall 110 and the second wall 112 to define a chamber interior having a chamber volume 116 and configured to contain a liquid 120, wherein a ratio of the first wall 110 surface area to chamber volume 116 is at least about 19 mm⁻¹; wherein at least a portion of at least one of the first wall 110 and second wall 112 is gas permeable. In certain preferred embodiments, the side wall of the incubation chamber may comprise either a curved line, a series of straight lines, or some combination of the two such that the cross-sectional shape of the incubation chamber 108 parallel to the first wall 110 is either curved, rectilinear, curvilinear or wedge-shaped.

In some embodiments, the body portion 102 of the microfluidic cartridge 100 may comprise a polymer. Examples of polymers that make up the body portion 102 may include but are not limited to: poly(methyl methacrylate) (PMMA), polycarbonate, polyethylene, polypropylene, polystyrene, polyesters, polyvinyl chloride (PVC), cyclic olefin polymer (COP), cyclic olefin copolymer (COC) and nylon.

### Breathable Membrane

In one of its aspects, the present invention provides a microfluidic cartridge for growing a microorganism in liquid culture wherein the microfluidic cartridge may comprise (a) a body portion having a mounting portion configured to be secured with respect to a driving apparatus; and (b) at least a first incubation chamber disposed in the body portion of the first incubation chamber comprising (i) a first wall, (ii) a second wall opposed to the first wall, and (iii) at least one sidewall interconnecting the first wall and the second wall to define a chamber interior having a chamber volume and configured to contain a liquid, wherein at least a portion of at least one of the first wall and second wall is gas permeable. By way of non-limiting example, in certain preferred embodiments, either the first wall of the incubation chamber, the second wall of incubation chamber or both may be gas permeable to permit a flow of gas into and out of the incubation chamber. This gas permeability may be accomplished by sealing the incubation chamber on the first wall, second wall, or both with a breathable membrane. FIG. 6 illustrates an exploded view of one embodiment of microfluidic cartridge assembly with a plurality of incubations chambers 108 disposed on the body portion 102 of the cartridge 100, wherein each incubation chamber 108 comprises a first wall 110, a second wall 112, and at least one sidewall 114 interconnecting the first wall 110 and the second wall 112 to define a chamber interior. In certain embodiments the first wall 110 may comprise a breathable membrane. In some embodiments the second wall 112 may comprise a breathable membrane. By way of non-limiting example, in certain embodiments, the breathable membrane may be any biocompatible, polymer film that is gas permeable, liquid and microbe impermeable. In some embodiments, the breathable membrane may be adhesive-backed. In some embodiments, the permeable membrane may be a gas-permeable thermopolymer. In some embodiments, the permeable membrane may be fabricated from a copolymer such as polyester-polyurethane copolymer or polyether-polyurethane copolymer.

In some embodiments, the membrane may be a clear, gas permeable biaxially-oriented polyethylene terephthalate film attached using an adhesive. In some embodiments, the breathable membrane may be attached only on one side of the body portion of the microfluidic cartridge. In other embodiments, the breathable membrane may be attached to both sides of the body of the microfluidic cartridge. In certain preferred embodiments, the membrane may be a flexible membrane. In other embodiments, the membrane may be a non-flexible membrane.

The addition of a breathable, gas-permeable membrane allows for sample aeration so that bacteria samples have access to atmospheric gases (e.g., oxygen) for growth. Moreover, the breathable sealing membranes also allow respiration, cell viability and cell growth to be maintained in leak-proof incubation chambers since the membrane does not peel and is impervious to liquids. In fact, many cellular-based assays depend upon continuing respiration for accuracy and reproducibility of the assays, and an extended period of ongoing cellular metabolism may be required for cells held in such plates. Membranes of the present disclosure assure uniformity of gas exchange and thus cellular respiration from chamber-to-chamber and sample-to-sample across the cartridge. This uniformity is important for experimental accuracy and valid comparisons among different cell samples held in different chambers within a cartridge.

In some embodiments, when a microfluidic cartridge comprises a gas permeable thermopolymer from which the body of the cartridge is molded, the microfluidic cartridge itself may function as a suitable breathable membrane.

In certain preferred embodiments, the permeable membranes may be of a thickness such that they are impervious to microorganisms and allow for sufficient oxygen permeability through the membrane. Consequently, when applied and adhered to an incubation chamber as described herein, microbial contaminants are likewise excluded from the sample chambers of the cartridge. The amount of gas permeability necessary depends on experimental design.

### Motor

In one of its aspects, the present invention provides a system for growing a microorganism in liquid culture comprising (a) a driving apparatus configured to house and oscillate a microfluidic cartridge; and (b) a microfluidic cartridge secured with respect to the driving apparatus, the microfluidic cartridge comprising: a body portion and at least a first incubation chamber comprising (i) a first wall, (ii) a second wall opposed to the first wall, and (iii) at least one sidewall interconnecting the first wall and the second wall to define a chamber interior having a chamber volume and configured to contain a liquid, wherein a ratio of the first wall surface area to chamber volume is at least about 19 mm⁻¹; wherein at least a portion of at least one of the first wall and second wall is gas permeable to facilitate a flow of gas into and out of the chamber interior.

In certain preferred embodiments, the driving apparatus may comprise a direct current (DC) motor. In some embodiments, the DC motor is brushless, while in other embodiments, the DC motor may be brush motor. Examples of DC motors may include but are not limited to stepper motors or servo motors.

In certain preferred embodiments, the motor may be configured such that the driving apparatus oscillates the incubation chamber back an forth at a predetermined frequency. By way of non-limiting example, the predetermined oscillation frequency may be between about 1 and 5 Hz. In certain preferred embodiments, the oscillation frequency may be about 4 Hz. In other preferred embodiments, the oscillation frequency may be about 2 Hz.

In some embodiments, the motor may be configured such that the driving apparatus oscillates the incubation chamber with an oscillation angle in a range of from 30 degrees and 330 degrees. In some embodiments, the motor may be configured to oscillate with an oscillation angle in a range of from about 30 degrees or about 40 degrees or about 50 degrees or about 60 degrees or about 70 degrees or about 80 degrees or about 90 degrees or about 100 degrees or about 110 degrees or about 120 degrees or about 130 degrees or about 140 degrees or about 150 degrees or about 160 degrees or about 170 degrees to about 180 degrees or about 190 degrees or about 200 degrees or about 210 degrees or about 220 degrees or about 230 degrees or about 240 degrees or about 250 degrees or about 260 degrees or about 270 degrees or about 280 degrees or about 290 degrees or about 300 degrees or about 310 degrees or about 320 degrees or about 330 degrees.

In some embodiments, the motor may be configured such that the driving apparatus oscillates the incubation chamber with an oscillation angle in a range of from 150 degrees and 210 degrees. In some embodiments, the motor may be configured such that the driving apparatus oscillates the incubation chamber with an oscillation angle in a range of from 30 to 330 degrees, or from 100 degrees to 260 degrees.

In some embodiments, the motor is configured such that the driving apparatus oscillates the incubation chamber at an angular acceleration in a range of about 100 rad/s² or about 120 rad/s² or about 140 rad/s² or about 160 rad/s² or about 180 rad/s² or about 200 rad/s² or about 220 rad/s² or about 240 rad/s² or about 260 rad/s² or about 280 rad/s² to about 300 rad/s² or about 320 rad/s² or about 340 rad/s² or about 360 rad/s² or about 380 rad/s² or about 400 rad/s² or about 420 rad/s² or about 440 rad/s² or about such that the driving apparatus oscillates the incubation chamber at an angular acceleration in a range of 100 to 500 rad/s². In some embodiments, the motor is configured such that the driving apparatus oscillates the incubation chamber at an angular acceleration in a range of 200 to 300 rad/s².

### Additional Elements

In certain preferred embodiments, the system and methods for growing a microorganism in liquid culture described herein may further comprise an incubator configured to incubate a microorganism in a microfluidic cartridge. By way of non-limiting example, in certain preferred embodiments the incubator may comprise a heating element. The heating element may comprise metal heating elements (i.e. iron/chromium/aluminum (FeCrAl) wires, nickel/chrome (Ni/Cr) 80/20 wires, copper/nickel (Cu/Ni) wires). In some embodiments, the heating element may comprise ceramic heating elements (i.e. MoSi2, PTC ceramics). In some embodiments, the heating element may comprise polymer PTC heating elements (i.e. PTC rubber material). In some embodiments, the heating element may comprise composite heating elements.

### Methods of Increasing Growth of a Microorganism and Further Processing

In yet another of its aspects, the present invention provides methods of growing a microorganism in liquid culture. Methods of growing a microorganism in liquid culture may comprise: (a) disposing a microorganism and a suitable growth medium in a first incubation chamber, wherein the incubation chamber comprises (i) a first wall, (ii) a second wall opposed to the first wall, and (iii) at least one sidewall interconnecting the first wall and the second wall to define a chamber interior having a chamber volume and configured to contain a liquid, wherein a ratio of the first wall surface area to chamber volume is at least about 19 mm⁻¹, wherein at least a portion of at least one of the first wall and second wall is gas permeable; and (b) mixing the microorganism and the growth medium by oscillating the incubation chamber back and forth along an oscillation path at a predetermined oscillation frequency.

FIG. 4 shows a non-limiting embodiment of a microfluidic cartridge 100, including an exemplary oscillation path 126 and oscillation protocol, in accordance with some aspects of the present disclosure. FIG. 4 illustrates an incubation chamber 108 disposed on the body portion 102 of a microfluidic cartridge 100. As shown by FIG. 4, when the microfluidic cartridge 100 is oscillated using the driving apparatus, the incubation chamber 108 is moved along an oscillation path 126 to a second position 122. The angle of oscillation 124 is defined as the angle between the incubation chamber 108 at starting point of the oscillation path 126 and the second position 122 of the incubation chamber at the end of the oscillation path 126.

FIG. 5 illustrates a cross-sectional view providing a more detailed view of an exemplary incubation chamber in a microfluidic cartridge, in accordance with some aspects of the present disclosure. As shown in FIG. 5, in certain preferred embodiments, the first incubation chamber 108 may comprise a first wall 110, a second wall 112 opposed to the first wall, and at least one sidewall 114 interconnecting the first wall 110 and the second wall 112 to define a chamber interior having a chamber volume 116 and configured to contain a liquid 120, wherein the liquid may be comprised of a microorganism and suitable growth medium. In certain preferred embodiments, when said liquid 120 is disposed in a first incubation chamber 108, it may occupy no more than ⅔ of the chamber volume 116, such that there remains a head space 118 within the incubation chamber. In certain preferred embodiments, the headspace 118 may be configured such that when the incubation chamber 108 is oscillated back and forth along an oscillation path, the head space 118 creates more surface area for gas exchange within the incubation chamber. By way of non-limiting example, the head space 118 may occupy between about ⅓ to about ½ of the total chamber volume 116.

In certain preferred embodiments, the methods disclosed herein for growing a microorganism in liquid culture may further comprise disposing a microorganism and a suitable growth medium in at least one additional incubation chamber, wherein the growth medium in the first incubation chamber comprises an anti-microbial agent free cell culture medium, and the growth medium in the at least one additional incubation chamber comprises comprising at least one anti-microbial agent.

In some embodiments, the anti-microbial agent is an antibiotic. Examples of antibiotics may include but are not limited to, a bactericidal antibiotic, a bacteriostatic antibiotic, a beta-lactam antibiotic, an aminoglycoside antibiotic, an ansamycin antibiotic, a macrolide antibiotic, a sulfonamide antibiotic, a quinolone antibiotic, an oxazolidinone antibiotic, a glycopeptide antibiotic, an anthraquinone antibiotic, an azole antibiotic, a nucleoside antibiotic, a peptide antibiotic, a polyene antibiotic, a polyether antibiotic, a steroid antibiotic, a tetracycline antibiotic, a dicarboxylic acid antibiotic, a metal or a metal ion antibiotic, a silver compound antibiotic, an oxidizing antibiotic or an antibiotic that releases free radicals or active oxygen, or a cationic antimicrobial agent.

In some embodiments, the methods disclosed herein comprise growing a microorganism in cell culture media. In some embodiments, the microorganism may be selected from the group of prokaryotic cells and eukaryotic cells. In some embodiments, the prokaryotic cells are Gram-negative bacteria. In some embodiments, the Gram-negative bacteria is selected from the group of Escherichia coli, Salmonella, Shigella, Enterobaceriaceae, Pseudomonas, Moraxella, Helicobacter, Strenotrophomonas, Bdellovibrio, and Legionella. In some embodiments, the prokaryotic cells are Gram-positive bacteria. In some embodiments, the Gram-positive bacteria is selected from the group of Enterococcus, Staphylococcus, Streptococcus, Actinomyces, Bacillus, Clostridium, Corynebacterium, Listeria, and Lactobacillus. In some embodiments, the eukaryotic cells are fungal cells. In some embodiments, the fungal cells are yeast. In some embodiments, the yeast is Candida.

In certain embodiments, methods of growing a microorganism in liquid culture may further comprise the step of incubating the microorganism by placing the incubation chamber in an incubator for a predetermined incubation period optimized to induce growth of the microorganism.

In some embodiments, incubating the microorganism may be conducted in a bacterial growth broth solution. By way of non-liming example, the bacterial growth broth solution may be a cation-adjusted broth solution, such as Mueller Hinton broth, lysogeny broth, super optimal broth, super optimal broth with catabolite repression, terrific broth, or M9 minimal broth.

In some embodiments, incubating the microorganism is conducted at a temperature in the range of 20° C. to 60° C. In some embodiments, incubating the microorganism is conducted at a temperature in the range of 30° C. to 50° C. In some embodiments, the microorganism may be incubated for at least 15, 30, 60, 90, 120, 150, 180, 210, 240, 270, 300, 360, 420, or 480 or more minutes. In some embodiments, incubating the microorganism is conducted at a temperature in the range of about 20° C., or about 21° C., or about 22° C., or about 23° C., or about 24° C., or about 25° C., or about 26° C., or about 27° C., or about 28° C., or about 29° C., or about 30° C. or about 31° C. or about 32° C. or about 33° C. or about 34° C. or about 35° C. or about 36° C. or about 37° C. or about 38° C. or about 39° C. to about 40° C. or about 41° C. or about 42° C. or about 43° C. or about 44° C. or about 45° C. or about 46° C. or about 47° C. or about 48° C. or about 49° C. or about 50° C., or about 51° C., or about 52° C., or about 53, ° C., or about 54° C., or about 55° C., or about 56° C., or about 57° C., or about 58° C., or about 59° C. or about 60° C.

In certain preferred embodiments, incubating the microorganism may be conducted at a temperature in the range of 33° C. to 47° C., or more preferably at a temperature in the range of 36° C. to 44° C.

In some embodiments, the microorganism may be incubated at room temperature e.g., about 25° C. In some embodiments, incubating the microorganism may be conducted at a temperature of about 37° C.

In some embodiments, the methods disclosed herein comprise a RiboGrow™ method. In some embodiments, the RiboGrow™ method is followed by lysis of the microorganism and release of a ribonucleic acid (RNA) molecule from the cells. In some embodiments, the cell lysate comprises an ribosomal RNA molecule. In some embodiments, the ribosomal RNA molecule is from a prokaryotic organism, or a fungal organism.

### Lysing

In some embodiments, the methods disclosed herein may further comprise lysing the microorganism to form a lysate. In certain preferred embodiments, lysis may include (a) subjecting a sample to mechanical lysis to cause disruption of a cellular membrane in the cellular material; (b) contacting the sample with an alkaline material to produce a lysate composition comprising the target chemical compound; and (c) recovering the lysate composition from the sample. Methods for lysing include those disclosed in International Patent Application No. PCT/US2018/045211, filed on Aug. 3, 2018, which is herein incorporated by reference in its entirety.

### Detection of a Nucleic Acid Molecule

In some embodiments, the methods disclosed herein further comprise detecting the quantity of a nucleic acid molecule from a microorganism in a sample. In some embodiments, the methods disclosed herein comprise comparing the quantity of a nucleic acid molecule in the antimicrobial agent-free inoculate to the quantity of a nucleic acid molecule in the antimicrobial agent inoculate.

In some embodiments, determining the quantity of a nucleic acid molecule in a plurality of inoculates comprises a sandwich assay. In some embodiments, determining the quantity of a nucleic acid molecule in a plurality of inoculates comprises using an electrochemical sensor platform.

In some embodiments, the buffer solution used to neutralize a cell lysate comprises a detector probe. In some embodiments a detector probe is added separately after a cell lysate is neutralized. In some embodiments, the detector probe comprises one or more nucleic acids. In some embodiments, the nucleic acids comprise one or more modified oligonucleotides. In some embodiments, the detector probe comprises a plurality of nucleic acids. In some embodiments, the detector probe comprises 1, 2, 3, 4, 5, 6, 7, 8, 9, 10, 11, 12, 13, 14, 15, 16, 17, 18, 19, 20 or more nucleic acids. In some embodiments, the detector probe comprises at least one deoxyribonucleic acid (DNA), peptide nucleic acid (PNA), locked nucleic acid (LNA), or any combination thereof. In some embodiments, the detector probe comprises one or more DNA. In some embodiments, the detector probe comprises a plurality of DNA. In some embodiments, the detector probe comprises 1, 2, 3, 4, 5, 6, 7, 8, 9, 10, 11, 12, 13, 14, 15, 16, 17, 18, 19, 20 or more DNA. In some embodiments, the detector probe comprises one or more PNAs. In some embodiments, the detector probe comprises a plurality of PNAs. In some embodiments, the detector probe comprises 1, 2, 3, 4, 5, 6, 7, 8, 9, 10, 11, 12, 13, 14, 15, 16, 17, 18, 19, 20 or more PNAs. In some embodiments, the detector probe comprises one or more LNAs. In some embodiments, the detector probe comprises a plurality of LNAs. In some embodiments, the detector probe comprises 1, 2, 3, 4, 5, 6, 7, 8, 9, 10, 11, 12, 13, 14, 15, 16, 17, 18, 19, 20 or more LNAs.

In some embodiments, the detector probe comprises a detectable label. In some embodiments, the detectable label is selected from a radionuclide, an enzymatic label, a chemiluminescent label, a hapten, and a fluorescent label. In some embodiments, the detectable label is a fluorescent molecule. In some embodiments, the fluorescent molecule is selected from a fluorophore, a cyanine dye, and a near infrared (NIR) dye. In some embodiments, the fluorescent molecule is fluorescein. In some embodiments, the fluorescent molecule is fluorescein isothiocyanate (FITC). In some embodiments, the detectable label is a hapten. In some embodiments, the hapten is selected from DCC, biotin, nitropyrazole, thiazolesulfonamide, benzofurazan, and 2-hydroxyquinoxaline. In some embodiments, the detectable label is biotin.

In some embodiments, the methods disclosed herein comprise contacting the neutralized cell lysate with a capture solution comprising a capture probe. In some embodiments, the capture probe comprises a capture sequence comprising a plurality of nucleic acids. In some embodiments, the nucleic acids comprise one or more modified oligonucleotides. In some embodiments, the capture probe comprises a plurality of nucleic acids. In some embodiments, the capture probe comprises 1, 2, 3, 4, 5, 6, 7, 8, 9, 10, 11, 12, 13, 14, 15, 16, 17, 18, 19, 20 or more nucleic acids. In some embodiments, the capture probe comprises at least one of deoxyribonucleic acid (DNA), peptide nucleic acid (PNA), locked nucleic acid (LNA), or any combination thereof. In some embodiments, the capture probe comprises DNA. In some embodiments, the capture probe comprises a plurality of DNA. In some embodiments, the capture probe comprises 1, 2, 3, 4, 5, 6, 7, 8, 9, 10, 11, 12, 13, 14, 15, 16, 17, 18, 19, 20 or more DNA. In some embodiments, the capture probe comprises one or more PNAs. In some embodiments, the capture probe comprises a plurality of PNAs. In some embodiments, the capture probe comprises 1, 2, 3, 4, 5, 6, 7, 8, 9, 10, 11, 12, 13, 14, 15, 16, 17, 18, 19, 20 or more PNAs. In some embodiments, the capture probe comprises one or more LNAs. In some embodiments, the capture probe comprises a plurality of LNAs. In some embodiments, the capture probe comprises 1, 2, 3, 4, 5, 6, 7, 8, 9, 10, 11, 12, 13, 14, 15, 16, 17, 18, 19, 20 or more LNAs. In some embodiments, at least a portion of the capture sequence is complementary to at least a portion of a nucleic acid molecule from the microorganism. In some embodiments, the capture probe further comprises a bead. In some embodiments, the bead is attached to the capture sequence. In some embodiments, the bead is a magnetic bead.

In some embodiments, the methods disclosed herein comprise contacting the neutralized cell lysate with a solution comprising streptavidin.

In some embodiments, the methods disclosed herein comprise detecting the quantity of a nucleic acid molecule from a microorganism in a sample. In some embodiments, the methods disclosed herein comprise comparing the quantity of a nucleic acid molecule in the antimicrobial agent-free inoculate to the quantity of a nucleic acid molecule in the antimicrobial agent inoculate. In some embodiments, the nucleic acid molecule is a deoxyribonucleic acid (DNA), ribonucleic acid (RNA), or a combination thereof.

In some embodiments, the methods disclosed herein further comprise a RiboResponse™ method. In some embodiments, the RiboResponse™ method comprises determining the quantity of an RNA molecule from the microorganism. In some embodiments, the RNA is a mature RNA. In some embodiments, the RNA is a precursor RNA. In some embodiments, the RNA is a ribosomal RNA (rRNA). In some embodiments, the rRNA is a 16S RNA or 23 S RNA. In some embodiments, the microorganism is a prokaryote. In some embodiments, the prokaryote is a Gram-negative bacterium. In some embodiments, the prokaryote is a Gram-positive bacterium. In some embodiments, the microorganism is fungal (e.g., candida).

The RiboResponse™ platform is quantitative in that more bacteria would result in more ribosomes and, hence, ribosomal RNA, resulting in a higher detection signal when ribosomal RNA is detected.

Methods for determining the quantity of an RNA molecule from the microorganism include those disclosed in International Patent Application No. PCT/US2018/047075, filed on Aug. 20, 2018, which is herein incorporated by reference in its entirety.

In some embodiments, when the methods disclosed herein comprise detecting the quantity of a nucleic acid molecule from a microorganism in a sample, the method can be completed in less than 4 hours or less, 3 hours or less, 2.5 hours or less, 2 hours or less, 90 minutes or less, 60 minutes or less, 45 minutes or less, or 30 minutes or less.

### Antimicrobial Agent Susceptibility

In some embodiments, the methods disclosed herein further comprise determining the susceptibility of a microorganism to an antimicrobial agent.

In some embodiments, in the methods and systems disclosed herein, at least one of the plurality of incubation chambers comprises at least one antimicrobial agent inoculate that comprises a microorganism in a cell culture media that contains an antimicrobial agent. In some embodiments, the plurality of inoculates comprises (a) at least one antimicrobial agent-free inoculate that comprises a microorganism in a cell culture media that does not contain an antimicrobial agent; (b) at least one antimicrobial agent inoculate that comprises a microorganism in a cell culture media that contains an antimicrobial agent; and (c) at least one antimicrobial agent inoculate that comprises a microorganism in a cell culture media that contains two antimicrobial agents. In some embodiments, the plurality of inoculates comprises (a) at least one antimicrobial agent-free inoculate that comprises a microorganism in a cell culture media that does not contain an antimicrobial agent; (b) 1, 2, 3, 4, 5, 6, 7, 8, 9, or 10 or more antimicrobial agent inoculates that each comprise a microorganism in a cell culture media that contains an antimicrobial agent; and (c) at least one antimicrobial agent inoculate that comprises a microorganism in a cell culture media that contains two antimicrobial agents. In some embodiments, the plurality of inoculates comprises (a) at least one antimicrobial agent-free inoculate that comprises a microorganism in a cell culture media that does not contain an antimicrobial agent; (b) at least one antimicrobial agent inoculate that comprises a microorganism in a cell culture media that contains an antimicrobial agent; and (c) 1, 2, 3, 4, or 5 or more antimicrobial agent inoculates that each comprise a microorganism in a cell culture media that contains two antimicrobial agents. In some embodiments, the cell culture media for at least 2, 3, 4, 5, 6, 7, 8, 9, or 10 or more antimicrobial agent inoculates contain different antimicrobial agents. In some embodiments, the cell culture media for at least 2, 3, 4, or 5 or more antimicrobial agent inoculates contain different combinations of antimicrobial agents.

In some embodiments, the microorganism is susceptible to the antimicrobial agent if the quantity of nucleic acid molecules of the microorganism in the antimicrobial agent-free inoculate is more than the quantity of nucleic acid molecules of the microorganism in an inoculate comprising the microorganism and the antimicrobial agent. In some embodiments, the microorganism is not susceptible to the antimicrobial agent if the quantity of nucleic acid molecules of the microorganism in the antimicrobial agent-free inoculate is nearly equal, equal, or less than the quantity of nucleic acid molecules of the microorganism in an inoculate comprising the microorganism and the antimicrobial agent.

### Reports and Data Transmission

In certain embodiments, the methods and systems disclosed herein may further comprise generating one or more reports. In some embodiments, the methods disclosed herein further comprise transmitting one or more reports. In some embodiments, the report includes information on the susceptibility of a microorganism to one or more antimicrobial agents or combinations of antimicrobial agents. In some embodiments, the report provides recommendations on a therapeutic regimen. In some embodiments, the report provides recommendations on the dosage of an antimicrobial agent.

### EXPERIMENTAL EXAMPLES

Embodiments of the present invention will now be illustrated with reference to the following examples which should not be used to construe or limit the scope of the present invention.

### Example 1

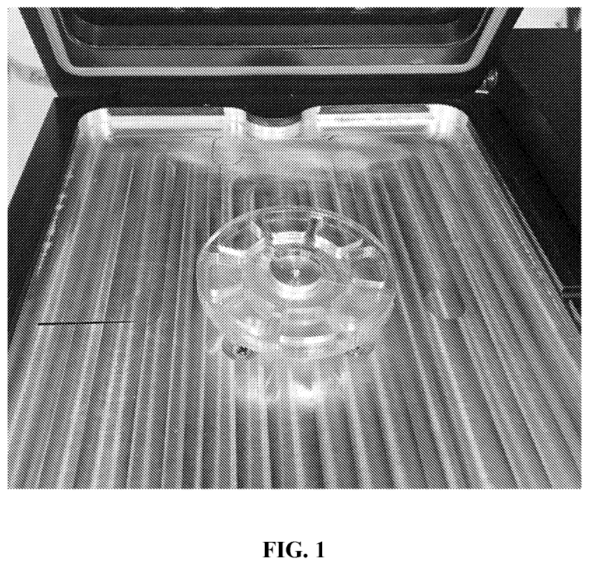

A Cook Medical MINC Benchtop incubator was modified to house a brushless DC motor and spinchuck. The motor was programmed to oscillate at an angular acceleration of 240 rad/s² and with an oscillation angle of 180 degrees. Microfluidic cartridges were laser cut from poly(methyl methacrylate) (PMMA) using a Trotec® Speedy 360 laser engraver. According to one example, the incubator and cartridge design are illustrated in FIG. 1. Specifically, FIG. 1 illustrates an interior view of the incubator spin-stand housing an incubation cartridge sealed with a breathable membrane using an adhesive positioned therebetween. A modified metal heating element is integrated into the incubator and positioned below the incubation cartridge. The incubation cartridge is placed on a spin-chuck with a DC motor integrated into the metal heat element.

FIG. 2 illustrates one example of the incubation cartridge design, where the cartridge includes eight incubation chambers and sample disposed in a portion of the eight incubation chambers. Disposed on both a first wall and the second wall of the incubation chambers of the incubation cartridge is a breathable membrane. The incubation chambers of the incubation cartridge were sealed with an adhesive-backed bio-compatible metal foil on both the top side and the bottom side of the cartridge to isolate and seal each chamber from each other.

Bacteria were cultured overnight by diluting 5 μL of stock E. coli glycerol with 5 mL of cation-adjusted Mueller Hinton (MH2) broth, diluted, recultured and rediluted to obtain a desired final concentration of 5×10⁵ colony-forming units per milliliter (CFU/mL). Two hundred microliters of the diluted bacteria-MH2 broth solution was added into each incubation chamber of the incubation cartridge or to a 96-well plate and immediately sealed with the half-breathable membrane.

The cultures were placed in either the modified incubator of FIG. 1 or in a tabletop shaker incubator. Cells were incubated at approximately 37° C. The modified incubator was operated at an angular acceleration/deceleration of 240 rad/s and 2300 rpm on the spinstand. The tabletop shaker incubator was operated at 400 rpm. Thereafter, 70 μL of sample was removed from the incubation chambers and the cells were lysed by incubating with 35 μL of 1M NaOH for about 5 minutes. The sample was then neutralized by adding 105 μL phosphate buffer solution.

Analysis was conducted on 150 μL of sample using a Luminex MagPix assay instrument with custom capture probes designed to hybridize with oligos on Luminex MagPlex-TAG microspheres. The total number of rRNA copies in the sample was determined at 0, 60, and 90 minute time intervals.

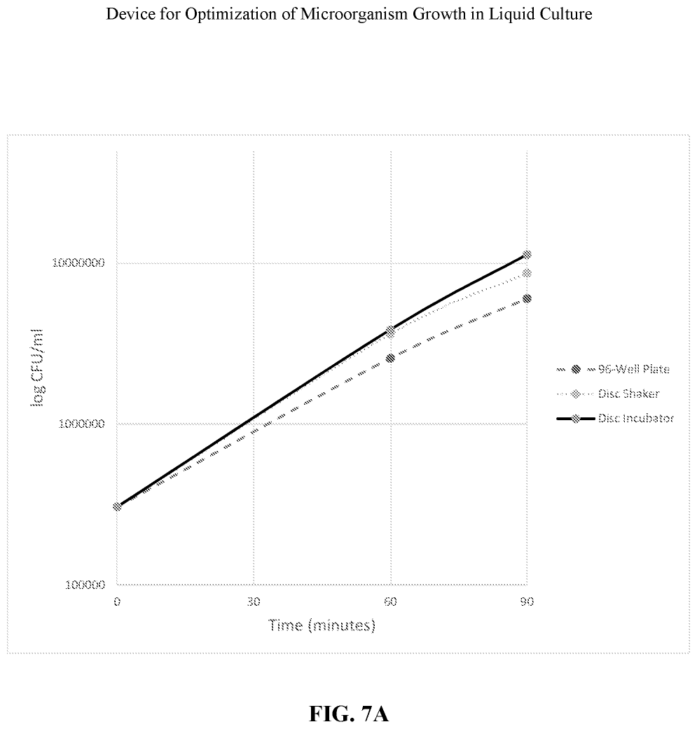

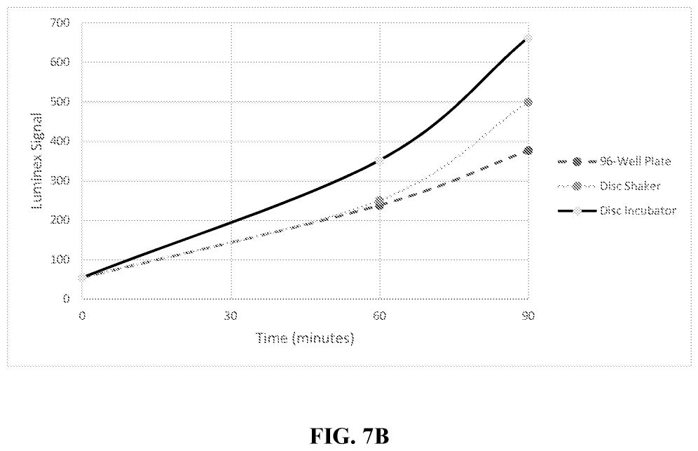

FIG. 7A compares the E. coli growth (in log CFU/mL) in the incubator cartridge in the incubator spinstand (see FIG. 1), the incubator cartridge in the plate shaker, and the standard 96-well plate in the plate shaker. FIG. 7B compares the Luminex signals of rRNA for E. coli grown in the incubator cartridge in the incubator spinstand (see FIG. 1), the incubator cartridge in the plate shaker, and the standard 96-well plate in the plate shaker. Fluidics within the cultures in the incubator spinstand exhibited more turbulence and advection than fluidics in the plate shaker incubator. By optimizing the mixing and aeration, an average 162% increase in RNA was seen in 90 minutes of incubation in the incubator spinstand compared with a 96-well plate on the plate shaker incubator. Furthermore, bacteria grown in the incubator cartridge in the plate shaker incubator showed an average 122% increase in RNA at 90 minutes in comparison to the 96-well plate, showing that both the type of mixing and aeration have an effect on bacterial reproduction.

### Example 2

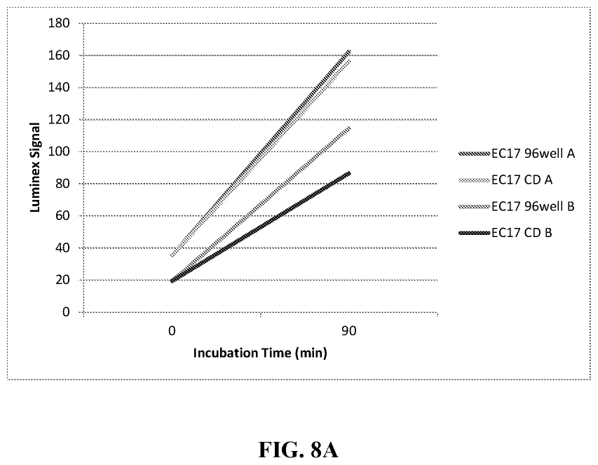

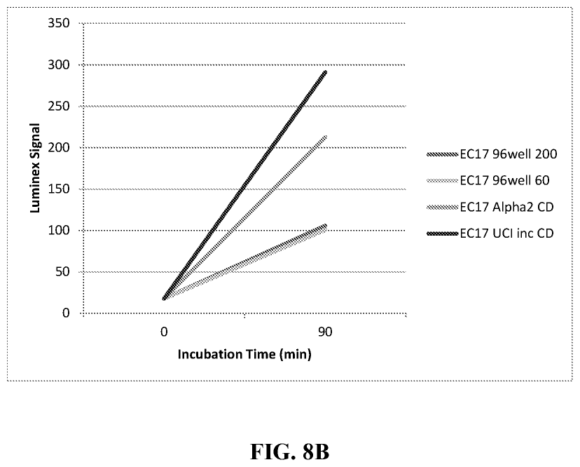

In this Example, using the relevant materials and methodology described in Example 1, bacterial were grown on two separate incubation cartridges in an incubator spinstand, and compared to a 96-well plate on an spin-stand incubator. The first incubation cartridge did not include an air permeable membrane while the second incubation cartridge did include an air permeable membrane. FIG. 8A shows the resulting Luminex signal results for bacteria grown in a microfluidic cartridge without a permeable membrane compared to the standard 96-well plate, while FIG. 8B shows the resulting Luminex signal results for bacteria grown in a microfluidic cartridge with a gas permeable membrane. As shown in FIG. 8B, based on the higher Luminex signal results, it is evident that using a permeable membrane in combination with a microfluidic incubation chamber provides increase microorganism growth over methods without a gas permeable membrane.

### Example 3

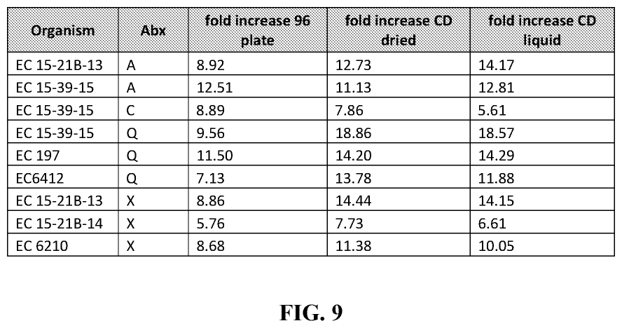

In this Example, using the relevant materials and methodology described in Example 1, bacteria were grown on an incubation cartridge in an incubator spinstand or in 96-well plate in a tabletop shaking incubator. Both the incubation cartridge and the 96-well plate included either liquid or dried down antibiotic agents (“Abx”) (e.g., ampicillin (“A”), cefazolin (“C”), ciprofloxacin (“Q”), or ceftriaxone (“X”)) in some chambers, in addition to some agent-free chambers. FIG. 9 shows the fold-increase from time 0 to 90 minutes of the resulting Luminex signals for different antibiotic resistant strains of E. coli grown in the presence of the various antibiotic agents. Based on these values shown in FIG. 9, the incubation cartridge consistently performs better than the 96-well plate for these bacteria grown with antibiotic agents.

The disclosure illustratively described herein can suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

While this invention has been described with reference to illustrative embodiments and examples, the description is not intended to be construed in a limiting sense. Thus, various modifications of the illustrative embodiments, as well as other embodiments of the invention, will be apparent to persons skilled in the art upon reference to this description. It is therefore contemplated that the appended claims will cover any such modifications or embodiments.

All publications, patents and patent applications referred to herein are incorporated by reference in their entirety to the same extent as if each individual publication, patent or patent application was specifically and individually indicated to be incorporated by reference in its entirety.

What is claimed is:

1. A system for growing a microorganism in liquid culture, comprising:
(a) a rotating platform on a driving apparatus; and
(b) at least one cartridge comprising a plurality of incubation chambers which rests upon said rotating platform, wherein said rotating platform provides turbulent mixing within the plurality of incubation chambers.

2. A system for growing a microorganism in liquid culture, comprising:
(a) a driving apparatus configured to house and oscillate a microfluidic cartridge; and
(b) a microfluidic cartridge secured with respect to the driving apparatus, the microfluidic cartridge comprising: a body portion and at least a first incubation chamber comprising (i) a first wall, (ii) a second wall opposed to the first wall, and (iii) at least one sidewall interconnecting the first wall and the second wall to define a chamber interior having a chamber volume and configured to contain a liquid, wherein a ratio of the first wall surface area to chamber volume is at least about 19 mm⁻¹;
wherein at least a portion of at least one of the first wall and second wall is gas permeable to facilitate a flow of gas into and out of the chamber interior.

3. The system of claim 2, wherein the microfluidic cartridge comprises a circular disc.

4. The system of claim 2 or 3, wherein a cross-section of the incubation chamber viewed through the first wall is curved.

5. The system of claim 2 or 3, wherein a cross-section of the incubation chamber viewed through the first wall is rectilinear.

6. The system of claim 2 or 3, wherein a cross-section of the incubation chamber viewed through the first wall is curvilinear.

7. The system of claim 2 or 3, wherein a cross-section of the incubation chamber viewed through the first wall is wedge-shaped.

8. The system of any of claims 2 to 7, wherein the first wall of the incubation chamber is gas permeable to permit a flow of gas into and out of the chamber interior.

9. The system of claim 8, wherein the first wall of the incubation chamber is configured to allow the introduction of oxygen bubbles into the incubation chamber.

10. The system of claim 8, wherein the first wall of the incubation chamber is configured to allow waste gases to be exhausted from the incubation chamber.

11. The system of any of claims 8 to 10, wherein the first wall of the incubation chamber comprises a breathable membrane.

12. The system of claim 11, wherein the breathable membrane comprises a biocompatible, polymer film that is gas permeable and liquid and microbe impermeable.

13. The system of claim 11, wherein the breathable membrane comprises a gas-permeable thermopolymer.

14. The system of claim 11, wherein the breathable membrane is fabricated from a material comprising copolymer.

15. The system of claim 14, wherein the copolymer comprises polyester-polyurethane copolymer or polyether-polyurethane copolymer.

16. The system of any of claims 2 to 7 wherein the second wall of the incubation chamber is gas permeable to permit a flow of gas into and out of the chamber interior.

17. The system of claim 16, wherein the second wall of the incubation chamber is configured to allow the introduction of oxygen bubbles into the incubation chamber.

18. The system of claim 16, wherein the second wall of the incubation chamber is configured to allow waste gases to be exhausted from the incubation chamber.

19. The system of any of claims 16 to 18, wherein the second wall of the incubation chamber comprises breathable membrane.

20. The system of claim 19, wherein the breathable membrane comprises a biocompatible, polymer film that is gas permeable and liquid and microbe impermeable.

21. The system of claim 19, wherein the breathable membrane comprises a gas-permeable thermopolymer.

22. The system of claim 19, wherein the breathable membrane is fabricated from a material comprising copolymer.

23. The system of claim 22, wherein the copolymer comprises polyester-polyurethane copolymer or polyether-polyurethane copolymer.

24. The system of any of claims 2 to 7, wherein both the first wall of the incubation chamber and the second wall of the incubation chamber are gas permeable to facilitate a flow of gas into and out of the chamber interior.

25. The system of claim 24, wherein the gas permeable first wall and second wall of the incubation chamber are configured to allow the introduction of oxygen bubbles in the chamber.

26. The system of claim 24, wherein the gas permeable first wall and second wall of the incubation chamber are configured to allow waste gases to be exhausted from the incubation chamber.

27. The system of any of claims 24 to 26, wherein the first wall and the second wall of the incubation chamber each comprises a breathable membrane.

28. The system of claim 27, wherein the breathable membrane comprises a biocompatible, polymer film that is gas permeable and liquid and microbe impermeable.

29. The system of claim 27, wherein the breathable membrane comprises a gas-permeable thermopolymer.

30. The system of claim 27, wherein the breathable membrane is fabricated from a material comprising copolymer.

31. The system of claim 30, wherein the copolymer comprises polyester-polyurethane copolymer or polyether-polyurethane copolymer.

32. The system of any of claims 2 to 31, wherein the microfluidic cartridge comprises a plurality of incubation chambers.

33. The system of claim 32, wherein the plurality of incubation chambers is integrally disposed in a common body portion of the cartridge.

34. The system of claim 32 or 33, wherein the plurality of incubation chambers are disposed annularly around a central axis on the microfluidic cartridge

35. The system of any of claims 32 to 34, wherein the plurality of incubation chambers is configured to oscillate in unison about the central axis.

36. The system of any of claims 32 to 35, wherein the plurality of incubation chambers are fluidically isolated from one another.

37. The system of any of claims 2 to 36, wherein the microfluidic cartridge further comprises at least one additional processing chamber disposed in the body portion of the microfluidic cartridge.

38. The system of claim 37 wherein the additional processing chamber is connected to the first incubation chamber by a microfluidic pathway on the microfluidic cartridge.

39. The system of claim 38, wherein the additional processing chamber is located upstream from the first incubation chamber.

40. The system of claim 38, wherein the additional processing chamber is located downstream from the first incubation chamber.

41. The system of any of claims 2 to 40, wherein the body of the microfluidic cartridge comprises a polymer.

42. The system of claim 41, wherein the polymer is selected from poly(methyl methacrylate) (PMMA), polycarbonate, polyethylene, polypropylene, polystyrene, polyesters, polyvinyl chloride (PVC), cyclic olefin copolymer (COC), cyclic olefin polymer (COP) and nylon.

43. The system of any of claims 2 to 42, wherein the driving apparatus is configured to oscillate the microfluidic cartridge in an arcuate oscillation path.

44. The system of claim 43, wherein the arcuate oscillation path has an oscillation angle of about 180 degrees.

45. The system of any of claims 2 to 42, wherein the driving apparatus is configured to oscillate the microfluidic cartridge in a linear oscillation path.

46. The system of any of claims 2 to 45, wherein driving apparatus is configured to oscillate the microfluidic cartridge at a predetermined oscillation frequency between 1 and 5 Hz.

47. The system of claim 46, wherein the predetermined oscillation frequency is 4 Hz.

48. The system of claim 46, wherein the predetermined oscillation frequency is 2 Hz.

49. The system of any of claims 2 to 48, wherein the driving apparatus is configured to oscillate the microfluidic cartridge at an angular acceleration in a range between 100 to 500 rad/s².

50. The system of any of claims 2 to 48, wherein the driving apparatus is configured to oscillate the microfluidic cartridge at an angular acceleration in a range between 150 to 210 rad/s².

51. The system of any of claims 2 to 50, further comprising an incubator comprising a heating element, wherein the heater may be used to incubate the microfluidic cartridge by subjecting the microfluidic cartridge to temperatures sufficient for growing microorganisms over a predetermined incubation period.

52. The system of claim 51, wherein said heating element comprises metal.

53. The system of claim 52, wherein the heating element is formed from a material comprising at least one of nickel/chrome (Ni/Cr), copper/nickel (Cu/Ni), or iron/chromium/aluminum (Fe/Cr/Al).

54. A method for growing a microorganism in a liquid culture comprising:
(a) disposing a microorganism and a suitable growth medium in a first incubation chamber, wherein the incubation chamber comprises (i) a first wall, (ii) a second wall opposed to the first wall, and (iii) at least one sidewall interconnecting the first wall and the second wall to define a chamber interior having a chamber volume and configured to contain a liquid, wherein a ratio of the first wall surface area to chamber volume is at least about 19 mm⁻¹, wherein at least a portion of at least one of the first wall and second wall is gas permeable; and
(b) mixing the microorganism and the growth medium by oscillating the incubation chamber back and forth along an oscillation path at a predetermined oscillation frequency.

55. The method of claim 54, further comprising the step of incubating the microorganism by placing the incubation chamber in an incubator for a predetermined incubation period.

56. The method of claim 55, wherein the incubator comprises a heating element.

57. The method of claim 56, wherein the heating element comprises metal.

58. The method of claim 56 or 57, wherein the heating element is formed from a material comprising at least one of nickel/chrome (Ni/Cr), copper/nickel (Cu/Ni), or iron/chromium/aluminum (Fe/Cr/Al).

59. The method of any of claims 54 to 58, further comprising disposing a microorganism and a suitable growth medium in at least one additional incubation chamber.

60. The method of claim 59, wherein the growth medium in the first incubation chamber comprises an anti-microbial agent free cell culture medium, and the growth medium in the at least one additional incubation chamber comprises at least one anti-microbial agent.

61. The method of claim 60, wherein the anti-microbial agent is an antibiotic.

62. The method of any of claims 54 to 61, further comprising incubating the microorganism in a bacterial growth broth solution.

63. The method of claim 62, wherein the bacterial growth broth solution is a cation-adjusted broth solution.

64. The method of any of claims 54 to 63, further comprising the step of introducing gas into the incubation chamber during mixing.

65. The method of claim 64, wherein the step of introducing gas into the incubation chamber is accomplished by passing gas through a gas permeable portion of the first wall of the incubation chamber.

66. The method of claim 64, wherein the step of introducing gas into the incubation chamber is accomplished by passing gas through a gas permeable portion of the second wall of the incubation chamber.

67. The method of any of claims 54 to 66, further comprising the step of exhausting waste gases from the incubation chamber during mixing.

68. The method of claim 67, wherein the step of exhausting waste gases from the incubation chamber is accomplished by passing waste gases through a gas permeable portion of the first wall of the incubation chamber.

69. The method of claim 67 wherein the step of exhausting waste gases from the incubation chamber is accomplished by passing waste gases through a gas permeable portion of the second wall of the incubation chamber.

70. The method of any of claims 54 to 69, wherein the oscillation path is an arcuate path.

71. The method of claim 70, wherein the arcuate path has an oscillation angle between 100 and 260 degrees.

72. The method of claim 70, wherein the arcuate path has an oscillation angle of about 180 degrees.

73. The method of any of claims 54 to 69 wherein the oscillation path is linear.

74. The method of any of claims 54 to 73, wherein the predetermined oscillation frequency is between 1 and 5 Hz.

75. The method of claim 74, wherein the predetermined oscillation frequency is 4 Hz.

76. The method of claim 74, wherein the predetermined oscillation frequency is 2 Hz.

77. The method of any of claims 54 to 76, wherein the incubation chamber is oscillated at an angular acceleration in a range between 100 to 500 rad/s².

78. The method of any one of claims 54 to 77, wherein the microorganism is bacteria.

79. The method of any one of claims 54 to 78, wherein the microorganism is gram-positive.

80. The method of any one of claims 54 to 78, wherein the microorganism is gram-negative.

81. The method of any one of claims 54 to 77, wherein the microorganism is fungal.

82. The method of any one of claims 54 to 81, wherein the microorganism and suitable growth medium when disposed in a first incubation chamber occupy no more than ⅔ of the chamber volume, such that there remains a head space within the incubation chamber.

83. The method of claim 82, wherein the headspace is configured such that when the incubation chamber is oscillated back and forth along an oscillation path, the head space creates more surface area for gas exchange within the chamber.

84. The method of claim 82 or 83, wherein the head space is between ⅓ to ½ of the total chamber volume.

85. A microfluidic cartridge for growing a microorganism in liquid culture comprising:
(a) a body portion having a mounting portion configured to be secured with respect to a driving apparatus;
(b) at least a first incubation chamber disposed in the body portion of the first incubation chamber comprising (i) a first wall, (ii) a second wall opposed to the first wall, and (iii) at least one sidewall interconnecting the first wall and the second wall to define a chamber interior having a chamber volume and configured to contain a liquid, wherein a ratio of the first wall surface area to chamber volume is at least about 19 mm⁻¹;
wherein at least a portion of at least one of the first wall and second wall is gas permeable.

86. The apparatus of claim 85, wherein the microfluidic cartridge comprises a circular disc.

87. The apparatus of claim 85 or 86, wherein a cross-section of the incubation chamber viewed through the first wall is curved.

88. The apparatus of claim 85 or 86, wherein a cross-section of the incubation chamber viewed through the first wall is rectilinear.

89. The apparatus of claim 85 or 86, wherein a cross-section of the incubation chamber viewed through the first wall is curvilinear.

90. The apparatus of claim 85 or 86, wherein a cross-section of the incubation chamber viewed through the first wall is wedge-shaped.

91. The apparatus of any of claims 85 to 90, wherein the first wall of the incubation chamber is gas permeable to permit a flow of gas into and out of the chamber interior.

92. The apparatus of claim 91, wherein the first wall of the incubation chamber is configured to allow the introduction of gas bubbles into the incubation chamber.

93. The apparatus of claim 91, wherein the first wall of the incubation chamber is configured to allow waste gases to be exhausted from the incubation chamber.

94. The apparatus of any of claims 91 to 93, wherein the first wall of the incubation chamber comprises a breathable membrane.

95. The apparatus of claim 94, wherein the breathable membrane comprises a biocompatible, polymer film that is gas permeable and liquid and microbe impermeable.

96. The apparatus of claim 94, wherein the breathable membrane comprises a gas-permeable thermopolymer.

97. The apparatus of claim 94, wherein the breathable membrane is fabricated from a material comprising copolymer.

98. The apparatus of claim 97, wherein the copolymer comprises polyester-polyurethane copolymer or polyether-polyurethane copolymer.

99. The apparatus of any of claims 85 to 90 wherein the second wall of the incubation chamber is gas permeable to permit a flow of gas into and out of the chamber interior.

100. The apparatus of claim 99, wherein the second wall of the incubation chamber is configured to allow the introduction of gas bubbles into the incubation chamber.

101. The apparatus of claim 99, wherein the second wall of the incubation chamber is configured to allow waste gases to be exhausted from the incubation chamber.

102. The apparatus of any of claims 99 to 101, wherein the second wall of the incubation chamber comprises breathable membrane.

103. The apparatus of claim 102, wherein the breathable membrane comprises a biocompatible, polymer film that is gas permeable and liquid and microbe impermeable.

104. The apparatus of claim 102, wherein the breathable membrane comprises a gas-permeable thermopolymer.

105. The apparatus of claim 102, wherein the breathable membrane is fabricated from a material comprising copolymer.

106. The apparatus of claim 105, wherein the copolymer comprises polyester-polyurethane copolymer or polyether-polyurethane copolymer.

107. The apparatus of any of claims 85 to 90 wherein both the first wall of the incubation chamber and the second wall of the incubation chamber are gas permeable to facilitate a flow of gas into and out of the chamber interior.

108. The apparatus of claim 107 wherein the gas permeable first wall and second wall of the incubation chamber are configured to allow the introduction of gas bubbles in the chamber.

109. The apparatus of claim 107, wherein the gas permeable first wall and second wall of the incubation chamber are configured to allow waste gases to be exhausted from the incubation chamber.

110. The apparatus of any of claims 107 to 109, wherein the first wall and the second wall of the incubation chamber each comprises a breathable membrane.

111. The apparatus of claim 110, wherein the breathable membrane comprises a biocompatible, polymer film that is gas permeable and liquid and microbe impermeable.

112. The apparatus of claim 110, wherein the breathable membrane comprises a gas-permeable thermopolymer.

113. The apparatus of claim 110, wherein the breathable membrane is fabricated from a material comprising copolymer.

114. The apparatus of claim 113, wherein the copolymer comprises polyester-polyurethane copolymer or polyether-polyurethane copolymer.

115. The apparatus of any of claims 85 to 114, wherein the microfluidic cartridge comprises a plurality of incubation chambers.

116. The apparatus of claim 115, wherein the plurality of incubation chambers is integrally disposed in a common body portion of the cartridge.

117. The apparatus of claim 115 or 116, wherein the plurality of incubation chambers are disposed annularly around a central axis on the microfluidic cartridge

118. The apparatus of any of claims 115 to 117, wherein the plurality of incubation chambers is configured to oscillate in unison about the central axis.

119. The apparatus of any of claims 115 to 118, wherein the plurality incubation chambers are fluidly isolated from one another.

120. The apparatus of any of claims 85 to 119, wherein the microfluidic cartridge further comprises at least one additional processing chamber disposed in the body portion of the microfluidic cartridge.

121. The apparatus of claim 120 wherein the additional processing chamber is connected to the first incubation chamber by a microfluidic pathway on the microfluidic cartridge.

122. The apparatus of claim 121, wherein the additional processing chamber is located upstream from the first incubation chamber.

123. The apparatus of claim 121, wherein the additional processing chamber is located downstream from the first incubation chamber.

124. The apparatus of any of claims 85 to 123, wherein the body of the microfluidic cartridge comprises a polymer.

125. The apparatus of claim 124, wherein the polymer is selected from poly(methyl methacrylate) (PMMA), polycarbonate, polyethylene, polypropylene, polystyrene, polyesters, polyvinyl chloride (PVC), cyclic olefin polymer (COP), cyclic olefin copolymer (COC) and nylon.
